(19) United States
(12) Patent Application Publication
(10) Pub. No.: US 2003/0059844 A1
(43) Pub. Date: Mar. 27, 2003

(54) Apparatus and method for predicting rules of protein sequence interactions

(75) Inventors: Jonathan Richard Heal, London (GB); Jonathan Swinton, Cambridge (GB); Robert Geoffrey Cooper, Epsom (GB)

Correspondence Address: BURR & BROWN, PO BOX 7068, SYRACUSE, NY, 13261-7068, US

(73) Assignee: Proteom Limited, Cambridge (GB)

(21) Appl. No.: 10/217,957

(22) Filed: Aug. 13, 2002

(30) Foreign Application Priority Data
Aug. 15, 2001 (GB) 0119890.2

(51) Int. Cl. G01N 33/53; G06G 7/48; G06G 7/58; G06F 19/00; G01N 33/48; G01N 33/50

(52) U.S. Cl. 435/7.1; 702/19; 703/11

(57) ABSTRACT

This invention relates to the prediction of protein sequence interactions. A method and apparatus are described for discovering rules of protein sequence interactions using a machine learning approach. The system uses a database of known protein sequence interactions, an algorithm to asses predictive quality of rules on one or more subsets of a protein sequence interaction database and a technique for generating new rule sets for testing. The system aims to optimise a rule set (descriptors of protein sequence interactions) against one or more predetermined criteria. One or more pairs of protein sequences which are likely to interact are generated according to the rule sets thus generated.

### FIELD OF THE INVENTION

The present invention generally relates to the field of bioinformatics and more specifically machine learning approaches to predict protein sequence interactions. In a preferred form of the invention, a genetic algorithm is utilised to determine which pair-wise amino acid contacts between protein sequences are good rules for predicting protein sequence interactions.

### BACKGROUND OF THE INVENTION

With the availability of complete DNA sequences for many prokaryotic and eukaryotic genomes, and soon for the human genome itself, it is important to develop reliable proteome-wide approaches for a better understanding of protein sequence function (Fields et al, 1997). As elementary constituents of cellular protein sequence complexes and pathways, protein sequence-protein sequence interactions are key determinants of protein sequence function. Protein sequence-interaction mapping approaches generate functional information for large numbers of genes that are predicted from complete genome sequences. This information, released as databases available on the Internet, is likely to transform the way biologists formulate and then address their questions of interest.

Protein sequence interactions can be determined empirically by laboratory based methodologies. To this end there are several main techniques commonly employed in the field. Two-hybrid screens represent the favoured method of many researchers (Fields and Song, 1989). The two-hybrid system is a useful way to detect protein sequences that interact with a protein sequence of interest. In general, it is used primarily for initial identification of interacting protein sequences, not for detailed characterization of the interaction. Another commonly used technique to catalogue protein sequence interactions is to separate proteinaceous elements by two-dimensional gel electrophoresis, followed by identification using tandem mass spectrometry (Dove, 1999).

Due to the labour intensive nature of these methods for identification of protein sequence interactions, computational approaches to this problem have been sought. In general, these methods rely upon using the context of a known pair of interacting protein sequences from one genome to infer potential interactions between protein sequences in another genome. For example, it has been shown that protein sequences which are fused together into a single chain in one genome may represent potential interactants in another genome (Marcotte et al., 1999).

In another method, it is reported that protein sequences which have similar phylogenetic profiles may have similar function. Thus, two protein sequences with similar inheritance patterns (profiles) tend to be ‘functionally linked’ and may participate together in a structural complex or a biochemical pathway (Pelligrini et al., 1999). In yet another method, expression profiles of protein sequences in different tissues are surveyed and assessed for scenarios when pairs of protein sequences share the same profiles. Interactions may be inferred from such events (Eisenberg et al, 2000).

These methods all rely upon genomic context of a given protein sequence—none of these methods apply a rule to a given protein sequence by understanding interaction rules at the amino acid level.

Databases of known protein sequence interactions are now available over the Internet. Examples of these are the Database of Interacting Protein sequences at the University of California (dip.doe-mbi.ucla.edu), Helicobacter pylori database (Legrain et al., 2001), PathCalling database from Curagen (www.curagen.com). These data sources list empirical data collected during experiments described above. For the purpose of the current invention, the database needs to represent data from experiments where all protein sequence combinations were tested for interaction such that an interaction list contains accurate information about true negatives as well as true positives.

Genetic Algorithms

Genetic Algorithms (GAs) have four differences of principle to classical optimisation algorithms (Goldberg (1989)):

1. GAs use a coded representation of the parameters, rather than the parameters themselves.

2. GAs search with a population of solution vectors, rather than a single solution vector.

3. GAs exclusively use values of the function under study, and do not consider auxiliary information, such as the derivative.

4. GAs use probabilistic transition rules, rather than deterministic rules.

The function parameters are represented by a structure called a ‘chromosome’ representing a solution, which is referred to as an entity later in this description. GAs optimise how well a set of chromosomes perform with reference to a particular function, unlike other algorithms that optimise only a single solution. GAs cope well with complicated functions.

Genetic algorithms are used for a diverse range of applications within science, technology and medicine. In particular, within the field of bioinformatics, GA approaches may be applied to the problem of protein sequence structure prediction (Dandekar, 1992). Protein sequence structure prediction is analytically difficult to solve. The problem is thought to stem from the exponential nature of the conformational search space. The number of conformations of a protein sequence with N amino acid residues grows exponentially as γ^{N} where γ is the average number of conformations per residue (typically ˜10). This suggests that an algorithm would require an exponential time to search the whole conformational space for the native state. For these reasons ‘intelligent’ conformational search algorithms have become popular in structure prediction. Unlike gradient-based methods (Mackay et al, 1990) which tend to terminate at local minima, genetic algorithms ‘hop’ around the conformational space independent of local derivatives. A selection process focuses the search in low energy areas, whereas a recombination stage maintains exploration of the search space.

Protein Sequence Profiles

In some cases the sequence of an unknown protein is too distantly related to any protein sequence of known structure to detect its resemblance by overall sequence alignment, but it can be identified by the occurrence in its sequence of a particular cluster of residue types which is variously known as a pattern, motif, signature, or fingerprint (Lesk A M, 1900). These motifs arise because of particular requirements on the structure of specific region(s) of a protein sequence which may be important, for example, for their binding properties or for their enzymatic activity. These requirements impose very tight constraints on the evolution of those limited (in size) but important portion(s) of a protein sequence.

The use of protein sequence patterns (or motifs) to determine the function(s) of protein sequences is becoming very rapidly one of the essential tools of sequence analysis. To this end, databases of common protein sequence motifs for a variety of organisms have been made available over the internet. For example, the PROSITE database contains over 1500 patterns which characterise over 30000 different protein sequences from a variety of organisms (Bairoch, (1997)).

Protein sequence profiles are expressed by a pattern language. For example, the PROSITE database uses a nomenclature to express a motif such as:

[RK]-G-{EDRK}-[AGSCI]-[FY]-[LIVA]-x-[FYM]

This regular grammar expression can be interpreted by PROSITE search software against a library of sequences. Other pattern profile databases exist. Notably these include ProDom (Corpet et al., 2000) and Pfam (Bateman et al, 1999), and interPro (Apweiler R. et al 2001) which aims to be a superset of the others.

Hidden Markov Models

Hidden Markov Models (HMM) are a general statistical modelling technique for ‘linear’ problems such as sequences or time series and have been widely used in speech recognition for many years. More recently HMM's have been used to describe protein sequence and DNA profiles (Haussler et al, 1994, Durbin et al 1998).

A Hidden Markov Model (HMM) is a finite model that describes a probability distribution over an infinite number of possible sequences. The HMM is composed of a number of states, together with transition probabilities between states. In a typical profile HMM for a consensus local sequence alignment there is, for each column of the alignment, one match, one insert and one delete state, together with a begin state with an insert state and an end state. A match state corresponds to a match between a sequence and the profile, an insert state to the insertion of additional characters in the sequence, and a delete state to the loss of an alignment column from the profile. When the model is in one of the match states it emits an amino acid with a defined emission probability and then moves on to an insert state, a delete state, or the next match state. The model is parameterised by assigning probabilities to each of the possible transitions between states, and to the emission probabilities in each match state.

Given a parameterised profile HMM, an arbitrary sequence can be scored for its similarity to the profile HMM (and hence to the original multiple alignment). In addition, a cutoff may be defined at one or more levels of stringency, above which a score is considered to be a hit, and below which a miss.

One de-facto standard for the definition of a profile HMM is provided by the PROSITE database, where data relating to a profile HMM entry defines default (position-independent) values of all transition parameters, together with all position-specific transition values in order, and of thresholds. Other information is provided which is useful in interpreting the resulting match score. This standard is documented within the PROSITE database, at www.expasy.ch/txt/profile.txt. An alternative standard is used by the HMMER2.0 program (hmmer.wustl.edu), in which all transition probabilities are given explicitly.

One object of the present invention is to provide a genetic algorithm and system that can generate a set of entities, which represent pairs of protein sequences which are likely to interact, which is predictive of protein sequence interactions without relating to any genomic context of a given protein sequence.

Therefore, an objective of this system is to generate a set of pairs of protein sequences that may be used in a predictive manner.

The present invention aids the identification of specific protein-protein interactions, relevant to a particular tissue, stage or disease.

The method of the invention provides a list of potential interacting protein sequences. This is a shortlist whose members can be quickly assessed in the laboratory by conventional biophysical techniques, such as a biosensor, affinity chromatography, mass spectrometry or NMR for direct biological interaction. One of a pair of identified protein sequences which interact may then be deemed as a potential target for a drug so as to disrupt the interaction between the protein sequences. The method of the invention thus aids the selection potential pairs of interacting proteins such that, for example,

(i) new biochemical pathway information can be determined

(ii) protein sequences may be identified as drug targets.

### SUMMARY OF THE INVENTION

According to the present invention there is provided a method of selecting one or more pairs of protein sequences which are likely to interact comprising the steps of

a) associating a fitness value with each of a first set of entities, an entity comprising a sequence of genes representing corresponding properties of a first amino acid sequence and a second amino acid sequence, the associating step comprising the sub steps of

i) selecting an entity from said first set;

ii) generating a set of pairs of protein sequences which are represented by that entity such that a first protein sequence matches the first amino acid sequence property and the second protein sequence matches the second amino acid sequence property; and

iii) determining a fitness value to be associated with the selected entity in dependence upon the number of pairs of protein sequences in the generated set which are known to interact;

b) generating a new set of entities in dependence upon the fitness value associated with each entity;

c) repeating steps a) and b) using said new set as the first set of step a) until a score dependent upon the associated fitness values is greater than a predetermined threshold; and

d) generating a set of pairs of protein sequences represented by the entity set resulting from step c).

Preferably the new set generated at step b) contains a child entity created according to the sub step of

iv) selecting a pair of parent entities from the first set and creating a child entity which comprises a gene from each of the selected parent entities.

It is an advantage if a parent entity is selected at sub step iv) in dependence upon the fitness value determined at sub step iii).

In a preferred embodiment the child entity created at sub step iv) further comprises a first mutant gene which is not contained in the sequence of genes of either parent entity.

It is preferable if the new set contains entities from the original set, in order to allow the new set to ‘stay on track’, so in a preferred embodiment the new set generated at step b) contains an entity created according to the sub step of

v) selecting the entity from the original set in dependence upon the fitness value determined at sub step iii).

Ideally the pairs of protein sequences represented by each entity of the new set, together with the fitness value associated with each entity of the new set are stored on a computer readable medium for future use.

In one embodiment of the invention the elements are a representation of a single amino acid, in a second embodiment of the invention the elements are a representation of a subsequence of amino acids, which may be represented by a hidden markov model.

According to another aspect of the invention there is provided a computer readable medium carrying a computer program for implementing the method described above. A computer program for implementing the method is also provided.

According to another aspect of the invention there is provided a set of pairs of protein sequences being the product of the above method.

According to another aspect of the invention there is provided an apparatus for selecting a pair of protein sequences which interact comprising:

a set evaluator arranged to associate a fitness value with each of an first set of entities, an entity comprising a sequence of genes representing corresponding elements of a first amino acid sequence and a second amino acid sequence, the set evaluator comprising:

an entity selector arranged to select an entity from said first set;

a matching protein sequence pair generator arranged to generate a set of pairs of protein sequences which are represented by an entity such that a first protein sequence matches the first amino acid sequence and the second protein sequence matches the second amino acid sequence; and

an entity evaluator arranged to determine a fitness value to be associated with the selected entity in dependence upon the number of pairs of protein sequences in the generated set which are known to interact;

and a set generator arranged to generate a new set of entities in dependence upon the fitness value associated with each entity.

### BRIEF DESCRIPTION OF THE DRAWINGS

Embodiments of the invention will now be described, by way of example only, in which

FIG. 1 is a block diagram showing the functional elements of the present invention;

FIG. 2 is a flow chart illustrating the method of the present invention;

FIG. 3 is a flow chart illustrating the method of generating protein sequences which match a particular entity;

FIG. 4 is a flow chart illustrating the method of evaluating the performance of a particular entity;

FIG. 5 is a flow chart illustrating generation of a new entity set; and

FIG. 6 is an example of increasing scores during several iterations of the method of the invention.

### DETAILED DESCRIPTION OF THE INVENTION

In the following description, numerous specific details are set forth in order to prove a thorough understanding of the present invention. It will be obvious, however, to one skilled in the art that the present invention may be practiced without using these specific details. In other instances, well-known methods and structures have not been described in detail so as not to unnecessarily obscure the present invention.

The method of the present invention iteratively generates a new a set of entities from a first set of entities. In order for the method to operate it must be possible to determine a score for a set of entities, which represents how well that set achieves a desired result. The iterative process of the method of the present invention produces a new set of entities which tends to provide a better score than a previously generated (or the first) set of entities.

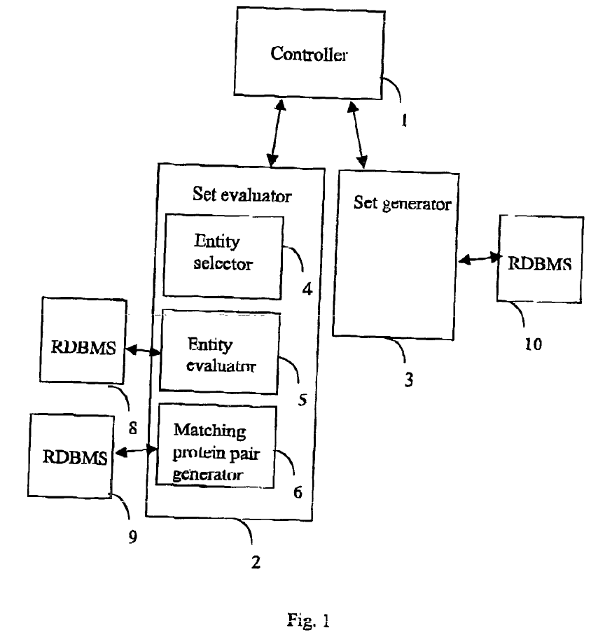

Referring to FIG. 1 an apparatus embodying the present invention comprises a controller 1, a set evaluator 2 and a set generator 3. The set evaluator 2 comprises an entity evaluator 4, an entity selector 5 and a matching protein sequence pair generator 6. A remote database management system (RDBMS) 9 contains information about pairs of protein sequences which are known to interact, and pairs of protein sequences which are known not to interact. Another RDBMS 10 is used to store information relating to entities and associated scores, which is generated by the entity evaluator 4. A third RDBMS 10 is used to store iteratively generated sets of entities. Furthermore the RDBMS 10 stores a set of genes which are available to the set generator 3. It will be understood that the databases 8, 9 and 10 may be different databases, or may be formed from different areas of the same database. Alternatively any computer readable storage media may be used.

An entity comprises a sequence of ‘genes’. Each gene represents corresponding properties of a first amino acid sequence, and a second amino acid sequence. In this embodiment a property comprises a single amino acid represented by a single letter code, so a gene is a representation of a pair of corresponding amino acids in a first and second amino acid sequence. The entity therefore comprises a sequence of pairs of letters, for example, [AS CT FR DF]. A particular amino acid may be represented in more than one gene in the sequence, and may be paired with any other gene, for example, [AS CT FR AP].

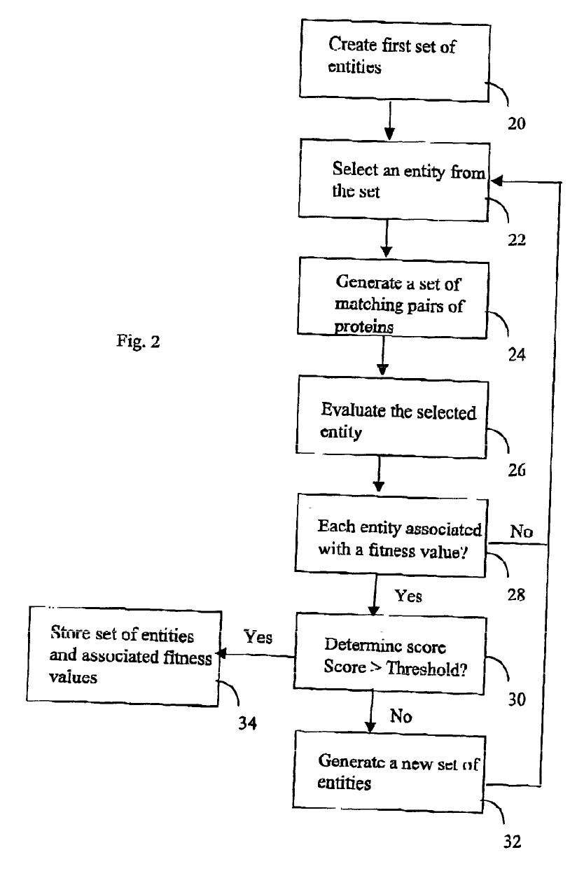

Referring now additionally to FIG. 2, at step 20 a first set of entities is created by the set generator 3 by randomly selecting a plurality of genes, which effectively form a gene pool, from the RDBMS 10, to form a first set of entities.

At step 22 an entity is selected from the set by the entity selector 5, for evaluation of the pair of amino acid sequences it represents. At step 24 a set of matching protein sequence pairs is generated by the matching protein sequence pair generator 6, by referring to a database of protein sequences which is stored in the RDBMS 9.

At step 26 the generated set of pairs of protein sequences is evaluated by the entity evaluator 4, with reference to stored data in the RDBMS 9 relating to known interaction of protein sequences, in order to determine a ‘fitness’ value to associate with the entity selected at step 22. Steps 22 to 26 are repeated until it is determined at step 28 that each entity of the entity set has a fitness value associated with it.

At step 30 a score, known as the objective function, for the entity set is calculated in dependence upon the fitness values associated with each entity in the entity set. In this embodiment of the invention the score is equal to the maximum fitness value associated with an entity in the entity set. If it the score greater than a predetermined threshold, which is determined at step 30, then the entities of the set together with the associated fitness values are stored in the RDBMS 9 at step 34.

If the predetermined threshold is not exceeded at step 30 then at step 32 a new set of entities is generated by the set generator 3, for evaluation in dependence upon the fitness values associated with each entity. Steps 22 to 32 are repeated until the predetermined threshold is exceeded at step 30.

The purpose of repeating steps 22 to 32 is to generate a set of entities such that the ability of the set to correctly predict known protein sequence interactions improves.

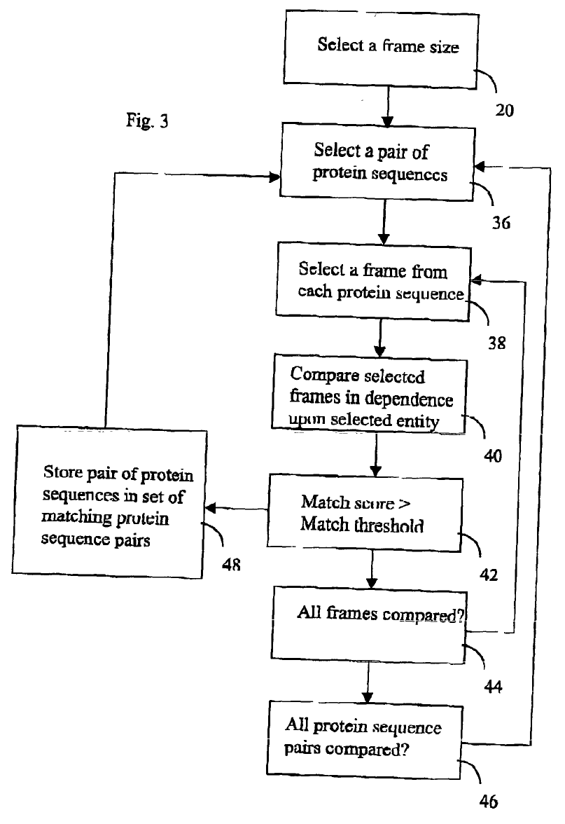

Referring now to FIG. 3 the generation step 24 will now be described in more detail. A frame size of between six and ten is selected at step 20 which determines the length of subsequence to be compared. A pair of protein sequences are selected from the RDBMS 9 at step 36. Frames comprising a subsequence of amino acids of the selected frame size are selected from each protein sequence and compared at step 40, in dependence upon the entity selected previously at step 22, to generate a match score. The match score is determined by the amino acid pairings encoded in the genes for the selected entity.

In this embodiment of the invention any match is scored equivalently. A pair of frames of equal length comprising a frame selected from a first protein sequence and a frame selected from a second protein sequence yields a score equal to the sum of amino acid pairs which match a gene in the given entity resulting from a sequential comparison of the elements in each of the pair of frames.

For example, if the selected entity comprises the genes [AS CT FR DF] then, a first frame comprising amino acid sequence APGFDOR would have a match score of 1 with a frame comprising amino acid sequence STRFANT, due to the gene AS. The first frame would have a match score of 3 with a frame comprising amino acid sequence TSTRFAN due to the genes GT, FR, DF. Matching pairs do not need to be sequential, so the first frame would have a match score of 4 with a frame comprising amino acid sequence SPTRFAN, as a sequential comparison of the frames results in a match with all of the genes in the entity. Furthermore, the order of the matching pairs is unimportant so a frame comprising the amino acid sequence RQDFGPA would have a match score of 4 with a frame comprising the amino acid sequence NAFRTPS. If the match score is greater than a predetermined match threshold, which is determined at step 42, then the pair of protein sequences selected at step 26 are stored in the generated set of matching protein sequence pairs at step 48, and new protein pair sequences are selected for comparison at step 36.

At step 44 a check is performed as to whether each possible combination of frames from the selected protein sequences have been compared to provide a match score. If not steps 36 to 42 are repeated.

Steps 26 to 44 are repeated until it is determined at step 46 that all of the pairs of protein sequences in the RDBMS 9 have been compared.

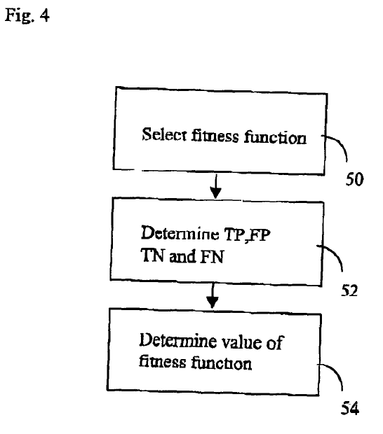

Referring now to FIG. 4 the evaluation step 26 will now be described in more detail. At step 50 a fitness function is selected from a set of possible fitness functions, the selected fitness function determines how the fitness score is calculated.

The possible fitness functions measure the ‘goodness of fit’ between known (empirically determined) interacting protein sequences and the set of pairs of protein sequences generated at step 24. The fitness functions are functions of

the number true positives (TP) which is the number of members of the generated set which are known to interact;

the number true negatives (TN) which is the number of pairs of protein sequences which are known not to interact and which are not in the generated set;

the number of false positives (FP) which are is the number of members of the generated set which are known not to interact; and

the number of false negatives (FN) which is the number of pairs of protein sequences which are known to interact and which are not in the generated set.

At step 52 TP, TN, FP and FN are determined, with reference to data stored in the RDBMS 9 which stores data about which protein sequences are known to interact.

In this embodiment of the invention the fitness functions are defined as follows:

1) PPV=TP/(FP+TP);

2) PPVOver2=TP/(FP+TP) where TP>2, otherwise −1;

3) PPVOver10=TP/(FP+TP) where TP>10, otherwise −1;

4) TPGoodPPV−TP when (FP+4)<TP, otherwise −1;

5) TPUnder50=TP when FP<50, otherwise −1;

6) Hamming=TN−TP;

At step 54 the selected fitness function is applied to the values calculated at step 52 to provide the fitness value for association with the entity selected at step 22 which is currently being evaluated.

The fitness value associated with each entity and the function used to calculate that fitness value are stored in the RDBMS 8 for audit trail purposes.

It can be seen that a different fitness function may be selected at step 50 each time the evaluation step 26 occurs. For example the PPVOver2 rule might be selected for initial iterations of the process, to select entities capable of accurately predicting a relatively small number of hits, and then for further iterations PPVOver10 may be selected.

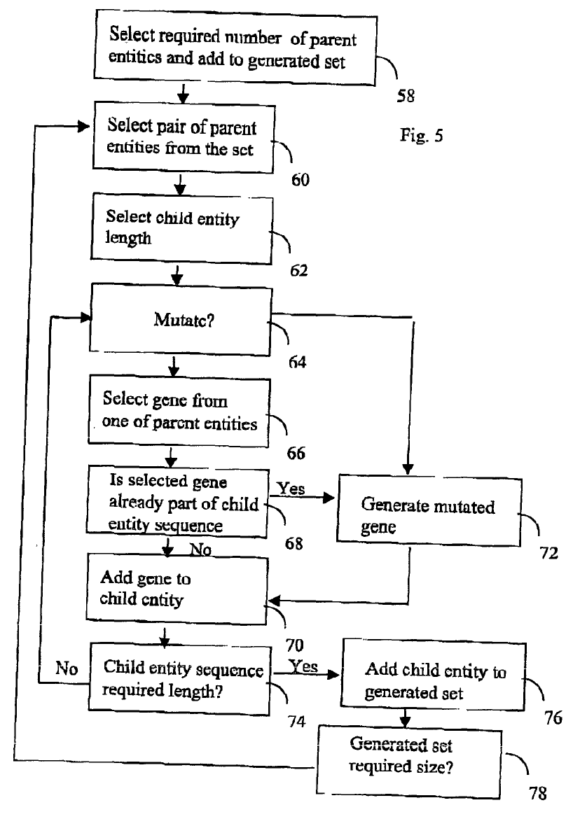

Referring now to FIG. 5 the new set generation step 32 will be described in more detail.

At step 58 a program variable, ‘parent retention value’ is used to determine the number of parent entities with the best associated fitness scores which are to be retained in the generated set. A non-zero value ensures that the generated set will retain at least one entity which scores as highly as the previous population. The parent retention value effectively manages the rate at which a newly generated set of entities ‘stays on track’. A number of entities equal to the parent retention value are selected from the entity set and added to the generated set.

At step 60 a pair of parent entities are selected from the set of entities. The parent entities are selected in dependence upon a probability, which is proportional to the fitness value of each entity.

A child entity is then created as follows. A sequence length for the child entity is selected at step 62. A program variable ‘chromosome length’ is set to control the length of the child entity and can be reset each time step 62 is performed to provide a random sequence length for each resulting child entity.

If a random mutation is not required as determined at step 64 then a gene is selected randomly from the gene sequence of one of the parents at step 66. The gene sequence of the child entity may not contain any duplicate genes. At step 68 a check is performed to determine whether the gene selected at step 66 already exists in the gene sequence of the child. If not, the selected gene is added to the gene sequence of the child entity at step 70.

If a gene that already exists in the gene sequence of the child entity is selected at step 66, then a mutation occurs at step 72 in which a new (mutated) gene, which is not in either of the gene sequences of the selected parent entities is created for addition to the child entity, at step 70. A new gene is created by selecting a gene randomly from a set of genes (the gene pool) which are stored in the RDBMS 10. In an alternative embodiment genes are not selected randomly from the set of genes, rather each gene is selected in turn from the set.

Mutation may occur randomly which is determined at step 64. A parameter ‘mutation rate’ controls the likelihood of a mutation occurring. If a mutation occurs at step 64 then steps 72 and 70 are performed, as described above.

Once a new entity is of correct size which is determined at step 74 the child entity is stored in a generated set. Otherwise steps 64 to 74 are repeated until the child entity sequence is of the required length.

Steps 60 to 76 are repeated until the generated set is of the required size, determined at step 78. The generated set is then stored in the RDBMS 10.

Therefore it will be appreciated that pairs of protein sequences which are likely to interact may be generated from a set of entities thus generated. It will also be appreciated that a set of protein sequences thus generated may conveniently be stored on compute readable media for use in further processes.

In another embodiment of the invention, the size of the child entity depends upon the gradient of fitness improvement.

Parameters and program variables mentioned above are set in the controller 1. These control constant values for a given learning cycle such as the size of the entity set, the parent retention value, the genes available for a mutation event, and parameters used during the generation step 24.

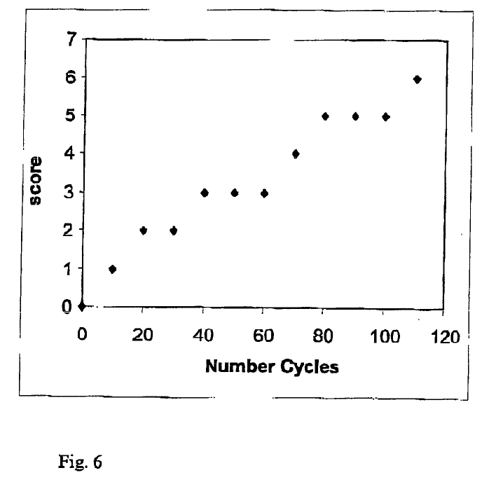

FIG. 6 illustrates the improvement in the score calculated at step 30 for an empirically derived protein sequence interaction data set for the Helicobacter pylori genome published in Nature, 2000 (4). The fitness function selected in this example was TPUnder50 .

The graph illustrates that for each iteration (of steps 22 to 32 in FIG. 1), the entity of maximum fitness value in each successively generated set produces successively better fitness scores. This typical output demonstrates that the method of this invention is able to generate sets of entities, which represent corresponding sequences of amino acids, which serve as good predictor of protein sequence interactions.

In other embodiments of the invention an element need not be a single amino acid, and may be a pattern profile identifier. For example, the following is a list of profile identifiers: PS01, PS02, PS03, PS04, PS05, PS06, PS07, PS08, PS09, PS10, which identify a pattern profile as described above.

A gene therefore comprises a pair of these identifiers, for example, (PS01, PS04) and an entity is composed of a sequences of genes such that we may have an entity containing 4 genes with the following definition ((PS01, PS04), (PS03, PS07), (PS05, PS04), (PS05, PS06))

The generation step 24 is modified accordingly. The input data set of protein sequences are searched for the presence or absence of each profile pairs defined by an entity. Presence of both pattern profiles defined by a gene within two protein sequences deems that gene a match.

Mutations are applied at step 72 by considering each element of a pattern profile (for a example [RK]-G-{EDRK}-[AGSCI]-[FY]-[LIVA]-x-[FYM]) and inserting into or deleting an amino acid from each with a specified probability (eg [FY]→[FYC] or (EDRK)→(ERK)), the x symbol interpreted as a full set of 20 amino acids. Alternatively, or in addition, elements may be deleted from the pattern (eg producing G-(EDRK)-[AGSCI]-[FY]-[LIVA]-x-[FYM]) from either end or inserted into the pattern at either end, either as a random sample from a set of possible elements or from a set elements known to have been previously deleted.

In another embodiment an element is an HMM, therefore a gene comprises a pair of profile HMMs.

The mutation step 72 is implemented as random variation in the values of individual transition and emission probabilities and/or score thresholds. Since the former are constrained to sum to one over the possible choices (of transitions and emissions respectively), after modification of any probability, that and all other probabilities of possible choices must be renormalized to sum to one. This is achieved by the division of each probability by the post-modification sum of all probabilities.

In this embodiment the additional mutational process may be provided such that new alignment columns may be inserted or deleted, with the consequent restructuring and reparameterisation of the profile HMM.

### REFERENCES

Apweiler R. et al (2001). Nucl. Acids Res. 29 (1): 37-40.

Bairoch, A., Ducher, P., Hofmann, K. (1997). The PROSITE database, its status in 1997. Nucleic Acids Research, 25 (1), 217-221.

Bateman A, Birney E, Durbin R, Eddy S R, Finn R D, Sonnhammer E L L (1999) Pfam 3.1: 1313 multiple alignments match the majority of proteins, Nucleic Acids Research 27:260-262

Corpet F, Servant F, Gouzy J, Kahn D (2000) ProDom and ProDom-CG: Tools for protein domain analysis and whole genome comparisons. Nucleic Acids Res. 28:267-269

Dandekar, T and Argos, P, “Potential of genetic algorithms in protein folding and protein engineering simulations”, Prot. Eng., 5, 637 (1992).

Dove, A (1999) Proteomics: translating genes into products? Nat. Biotechnol., 17, 233-236

Durbin, R., S. Eddy, A. Krogh, G. Mitchison. Biological Sequence Analysis. Cambridge U. Press, 1998

Eisenberg D, Marcotte E M, Xenarios I, Yeates T O, Protein function in the post-genomic era. Nature. 2000 Jun 15;405(6788): 823-6. Review.

Fields, S. The future is function. Nature Genet. 15, 325-327 (1997).

Fields, S and Song, O-K. (1989) Nature, 340, 245-246

Goldberg. Genetic algorithms in search, optimization, and machine learning. Reading, Mass.: Addison-Wesley, 1989.

Haussler D et al (1994) J Mol Biol 235, 1501 1531

Legrain et al (2001) Nature 409, 211-215

Lesk A. M. Computational Molecular Biology, Lesk A. M., Ed., pp17-26, Oxford University Press, Oxford (1988).

Mackay, A. J. Cross and A. T. Hagler, “The Role of Energy Minimization in Simulation Strategies of Biomolecular Systems”, in Prediction of protein structure and the principles of protein conformation, G. D. Fasman, ed., Plenum Press, 1990.

Marcotte, M. Pellegrini, M. J. Thompson, T. D. Yeates, and D. Eisenberg. A combined algorithm for genome-wide prediction of protein function. Nature 402, 83-86 (1999).

Pellegrini, E. M. Marcotte, M. J. Thompson, D. Eisenberg, and T. O. Yeates. Assigning protein functions by comparative genome analysis: Protein phylogenetic profiles. Proc. Natl. Acac. Sci. U.S.A. 96, 4285 (1999).

1. A method of selecting one or more pairs of protein sequences which are likely to interact comprising the steps of
a) associating a fitness value with each of a first set of entities, an entity comprising a sequence of genes representing corresponding properties of a first amino acid sequence and a second amino acid sequence, the associating step comprising the sub steps of:
iv) selecting an entity from said first set;
v) generating a set of pairs of protein sequences which are represented by that entity such that a first protein sequence matches the first amino acid sequence property and the second protein sequence matches the second amino acid sequence property; and
vi) determining a fitness value to be associated with the selected entity in dependence upon the number of pairs of protein sequences in the generated set which are known to interact;
b) generating a new set of entities in dependence upon the fitness value associated with each entity; and
c) repeating steps a) and b) using said new set as the first set of step a) until a score dependent upon the associated fitness values is greater than a predetermined threshold; and
d) generating a set of pairs of protein sequences represented by the entity set resulting from step c).

2. A method according to claim 1, in which the new set generated at step b) contains a child entity created according to the sup step of
iv) selecting a pair of parent entities from the first set and creating a child entity which comprises a gene from each of the selected parent entities.

3. A method according to claim 2, in which a parent entity is selected at sub step iv) in dependence upon the fitness value determined at sub step iii).

4. A method according to claim 2, in which the child entity created at sub step iv) further comprises a first mutant gene which is not contained in the sequence of genes of either parent entity.

5. A method according to claim 4, in which the first mutant gene is selected from a set of candidate genes.

6. A method according to claim 5, in which the child entity created at sub step iv) further comprises a second mutant gene selected from said set of candidate genes such that the second gene is not equal to the first mutant gene.

7. A method according to claim 1, in which the new set generated at step b) contains an entity created according to the sup step of
v) selecting the entity from the original set in dependence upon the fitness value determined at sub step iii).

8. A method according to claim 1, further comprising the step of
c) storing the pairs of protein sequences represented by each entity of the new set, together with the fitness value associated with each entity of the new set on a computer readable medium for future use.

9. A method according to claim 1, in which the elements are a representation of a single amino acid.

10. A method according to claim 1, in which the elements are a representation of a subsequence of amino acids.

11. A method according to claim 10 in which the representation of subsequence is a hidden markov model.

12. A method according to claim 9 in which the generating sub step ii) comprises the sub steps of
selecting a subsequence size corresponding to a number of amino acid sequence elements,
selecting a first protein sequence and a second protein sequence from a set of protein sequences;
comparing a subsequence of the first protein sequence with a subsequence of the second protein sequence by comparing a pair of elements at corresponding positions within each such pair of subsequence with a gene of the entity selected at sub step i) to generate a match score for the pair of subsequences;
adding the pair of protein sequences to the set of pairs of protein sequences if the match score is greater than a predetermined match score threshold

13. A computer readable medium carrying a computer program for implementing the method according to claim 1.

14. A computer program for implementing the method according to claim 1.

15. A set of pairs of protein sequences being the product of the method according to claim 1.

16. A set of entities representing pairs of protein sequences being generated at step c) of a method according to claim 1.

17. An apparatus for selecting a pair of protein sequences which interact comprising
a set evaluator arranged to associate a fitness value with each of an first set of entities, an entity comprising a sequence of genes representing corresponding properties of a first amino acid sequence and a second amino acid sequence, the set evaluator comprising
an entity selector arranged to select an entity from said first set;
a matching protein sequence pair generator arranged to generate a set of pairs of protein sequences which are represented by that entity such that a first protein sequence matches the first amino acid sequence property and the second protein sequence matches the second amino acid sequence property; and
an entity evaluator arranged to determine a fitness value to be associated with the selected entity in dependence upon the number of pairs of protein sequences in the generated set which are known to interact;
and a set generator arranged to generate a new set of entities in dependence upon the fitness value associated with each entity.

18. An apparatus according to claim 17, further comprising a memory for storing the pairs of protein sequences represented by each entity of the new set.

19. A drug arranged to target a protein sequence of a pair selected according to claim 1, such that interaction between said pair of protein sequences is disrupted.
